(19) United States
(12) Patent Application Publication
(10) Pub. No.: US 2018/0301206 A1
(43) Pub. Date: Oct. 18, 2018

(54) SYSTEMS, APPARATUS, AND METHODS FOR AN IMPROVED HEALTHCARE SERVICE SYSTEM

(71) Applicants: John Robbins, Lakeport, MI (US); Dan Robbins, St. Clair, MI (US); Bill Robbins, Fort Gratiot, MI (US); Dave Gao, Detroit, MI (US)

(72) Inventors: John Robbins, Lakeport, MI (US); Dan Robbins, St. Clair, MI (US); Bill Robbins, Fort Gratiot, MI (US); Dave Gao, Detroit, MI (US)

(21) Appl. No.: 15/838,352

(22) Filed: Dec. 11, 2017

Related U.S. Application Data
(60) Provisional application No. 62/431,823, filed on Dec. 9, 2016.

(51) Int. Cl. G16H 10/60; G06Q 20/14

(52) U.S. Cl. CPC G16H 10/60; G06Q 20/14

(57) ABSTRACT

An improved healthcare service includes a patient device adapted to provide interfaces for indicating whether a procedure is inpatient, entering codes and prices for the procedure, asking questions of the patient, create a payment summary, generate a payment, editing procedure descriptions, listing pending procedures and providers that can perform procedures, and displaying prices of services of a selected provider; a doctor device adapted to provide interfaces for displaying a listing of procedures of patients of a doctor; posting the doctor's available services and schedules; editing the doctor's specialty; posting needs for facilities and specialists; viewing incoming requests for services; searching for and communicating with facilities and specialists; and a server adapted to provide databases for storing information communicated between the patient and doctor device, provider information, information regarding diagnosis related groups of procedures, and information regarding procedure pricing; and determine pricing based on the databases. Numerous additional aspects are disclosed.

### RELATED APPLICATION

The present application claims priority to pending U.S. Provisional Application No. 62/431,823 filed Dec. 9, 2016, and titled “SYSTEMS, APPARATUS, AND METHODS FOR AN IMPROVED HEALTHCARE SERVICE SYSTEM” which is hereby incorporated herein by reference for all purposes.

### FIELD

The present invention relates to healthcare, and more specifically to systems, apparatus, and methods for an improved healthcare service system.

### BACKGROUND

The escalating costs of the existing U.S. healthcare system are well documented. Thirty-five percent of Americans have difficulty paying their medical bills, and nearly two-thirds of all bankruptcies are linked to inability to pay medical bills due to being uninsured or underinsured. Compounding the problem, thirty cents of every dollar spent on medical care in America is wasted, which amounts to $750 billion annually. Further, there is a severe lack of transparency with regard to costs in the existing system. There is more cost information readily available to compare and purchase a new car than there is to choose where to go for lifesaving healthcare. Only recently did the U.S. government release the prices that hospitals charge for the 100 most common medical procedures, revealing tremendous and seemingly random variation in the costs of services. For example, a hip replacement costs $5,300 in Ada, Okla., and $223,000 for exactly the same procedure in Monterrey Park, Calif. Thus, what is needed are improved systems, apparatus, and methods for an improved healthcare service system.

### SUMMARY

In some embodiments, the present invention provides an improved system for healthcare service. The system can include a (1) patient device including a processor coupled to a memory and a communications capability, the memory storing instructions executable by the processor, the instructions adapted to (A) provide a payment module including instructions to provide an interface for indicating whether a procedure is an inpatient procedure; provide an interface for entering codes and prices for the procedure; provide an interface for asking questions of the patient; create a payment summary; and generate a payment; and (B) provide a procedures module including instructions to provide an interface listing pending procedures; provide an interface for editing procedure descriptions; provide an interface listing providers that can perform a particular procedure; provide an interface for displaying prices of services of a selected provider; display other useful metrics of providers such as, for example, distance to provider and hospital infection rate; and display information from providers such as, for example, the availability of special schedules and special pricing; (2) a doctor device including a processor coupled to a memory and a communications capability, the memory storing instructions executable by the processor, the instructions adapted to provide an interface displaying a listing of procedures of patients of a doctor provide an interface for posting the doctor's available services and schedules; provide an interface for editing the doctor's specialty; provide an interface for posting needs for facilities and specialists; provide an interface for viewing incoming requests for services; provide an interface for searching for facilities and specialists; and provide an interface for communicating with facilities and specialists; and (3) a server including a processor coupled to a memory and a communications capability, the memory storing instructions executable by the processor, the instructions adapted to facilitate communications between the patent device and the doctor device; provide a first database for storing information communicated between the patent device and the doctor device; provide a second database for storing provider information; provide a third database for storing information regarding diagnosis related groups (DRGs) of procedures; provide a forth database for storing information regarding procedure pricing; and determine pricing based on information stored in the first through fourth databases.

Still other features, aspects, and advantages of the present invention will become more fully apparent from the following detailed description, the appended claims, and the accompanying drawings by illustrating a number of exemplary embodiments and implementations for carrying out the present invention. Embodiments of the present invention may also be capable of other and different applications, and its several details may be modified in various respects, all without departing from the spirit and scope of the present invention. Accordingly, the drawings and descriptions are to be regarded as illustrative in nature, and not as restrictive. The drawings are not necessarily drawn to scale. The description is intended to cover all modifications, equivalents, and alternatives falling within the spirit and scope of the claims.

### BRIEF DESCRIPTION OF THE DRAWINGS

FIG. 1 is a schematic block diagram depicting an example of a hardware system for an improved healthcare service system according to embodiments of the present invention.

FIG. 2 is a schematic block diagram depicting an example system architecture for an improved healthcare service system according to embodiments of the present invention.

FIG. 3 is a flowchart illustrating an example first application for patients in an improved healthcare service system according to embodiments of the present invention.

FIG. 4 is a flowchart illustrating an example second application for patients in an improved healthcare service system according to embodiments of the present invention.

FIG. 5 is a flowchart illustrating an example application for doctors in an improved healthcare service system according to embodiments of the present invention.

FIG. 6 is a flowchart illustrating an example application for healthcare facilities in an improved healthcare service system according to embodiments of the present invention.

FIG. 7 is a flowchart illustrating an example application for pricing in an improved healthcare service system according to embodiments of the present invention.

### DESCRIPTION

Embodiments of the present invention provide systems, apparatus, and methods for an improved healthcare service system. The existing U.S. healthcare system does not allow patients and their doctors to easily determine the most cost effective and rational procedures to select for treatment based on comparison of all available options. In addition, the billing and payment systems used in the existing healthcare system not only exacerbates the problem by contributing to the lack of transparency, but they also introduce delays and additional complexities that make it difficult for patients to know total costs upfront and for doctors to get timely compensated. Embodiments of the present invention address these issues by providing a system wherein patients are able to work with their doctors to determine/select procedures, facilities and costs via an online healthcare service “marketplace” and pay for them in advance.

Embodiments of the present invention provide a system that can be implemented as both an Internet website and an application that executes on a smartphone, tablet, laptop, PC or other device. The website and the application can include identical functionality and interfaces. Thus, reference to the application (e.g., App) herein will be understood to be a reference to both the application and website implementations unless otherwise stated.

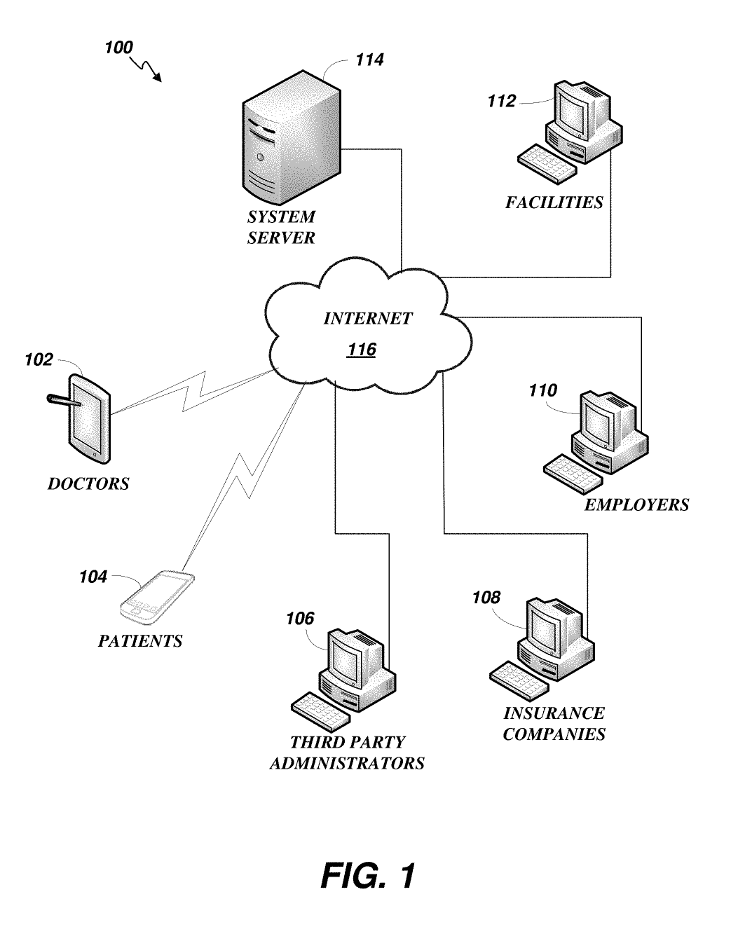

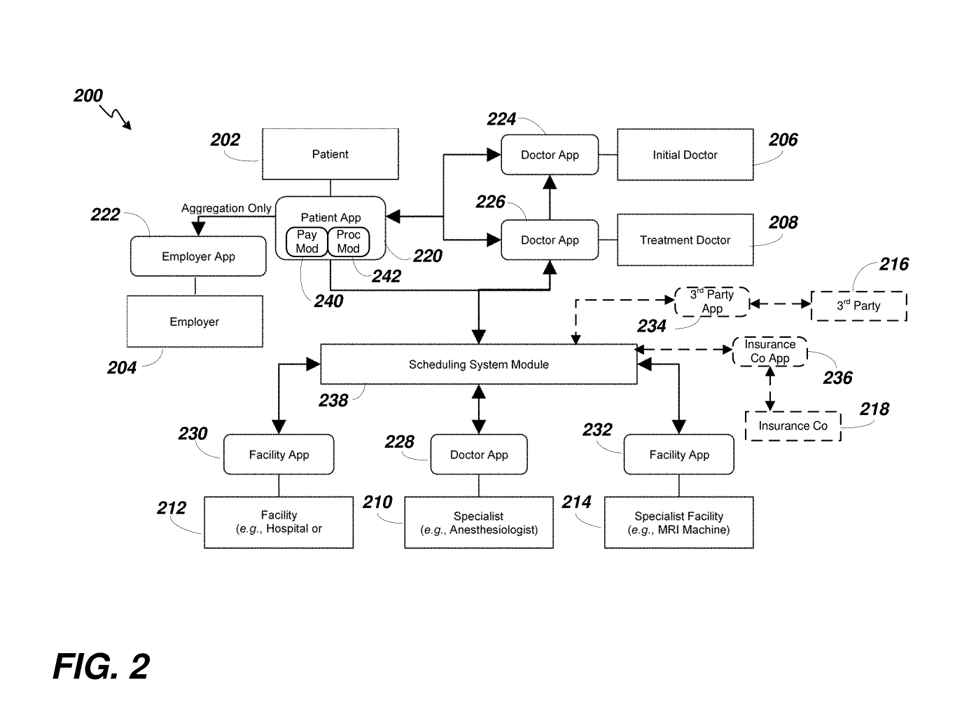

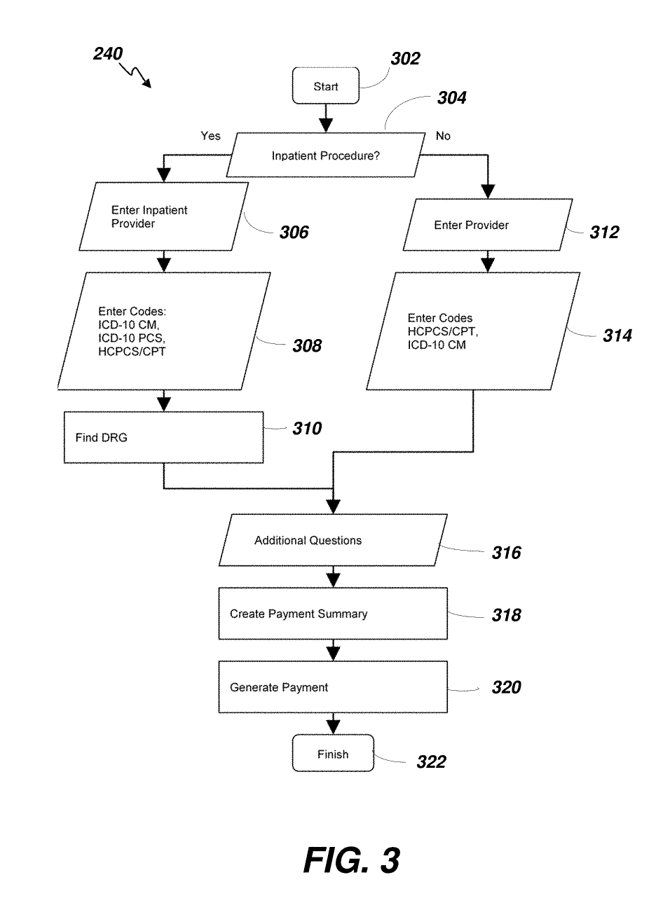

Turning now to FIG. 1, an example of the hardware system 100 of embodiments of the present invention is illustrated. Note that each node depicted in the data communication network of the system 100 can represent a plurality of devices that can independently communicate within the system 100. Further note that although for example, the doctors node 102 is represented with an image of a tablet and the patients node 104 is represented by an image of a smartphone, any suitable computing device could be used for any of the depicted nodes. As shown, the system 100 can include doctors nodes 102, patients nodes 104, third party nodes 106, insurance company nodes 108, employer nodes 110, facilities nodes 112, and system server nodes 114. The various nodes can be connected via local and/or wide area networks (e.g., via private networks, wireless networks, the Internet 116 (as shown in the example), etc.) so that the nodes can communicate as needed to perform the methods described below. The nodes can include the interfaces and databases described herein as well as software that implements the system architecture depicted in FIG. 2 and the methods depicted in FIGS. 3 to 7. The various users of the system 100 (described below as “actors”) and software components (e.g., apps or modules) correspond to and use/interact via the similarly named nodes of the system 100.

As shown in the system architecture diagram 200 of FIG. 2, in some embodiments, the systems and methods of the present invention can involve the following actors: the Patient 202 who is receiving and deciding on the treatment, the Employer 204 who, for example, will ultimately pay the majority of the price of the treatment, the Initial Doctor 206 who makes the diagnosis and creates the Procedure, the Treatment Doctor 208 who performs the Procedure, and a series of Specialists 210 and Facilities 212,214 that the Treatment Doctor 208 engages to help perform the Procedure. In other embodiments, third party administrators 216 and insurance companies 218 can also optionally be involved (as indicated by the dashed lines representing these actors in phantom).

As further shown in FIG. 2, the Patient 202 interacts with the system 100 via the Patient App 220 (which executes on the patients node 104, FIG. 1), the Employer 204 interacts with the system 100 via the Employer App 222 (which executes on the employers node 110, FIG. 1), the Initial Doctor 206 and the Treatment Doctor 208 who interact with the system 100 via separate instances of the Doctors App 224, 226 (which execute on different doctors nodes 102, FIG. 1), the Specialists 210 interact with the system 100 via yet additional instances of the Doctors App 228 (which execute on yet additional doctors nodes 104, FIG. 1), and the Facilities 212,214 (or managers thereof) interact with the system 100 via separate instances of the Facilities App 230, 232 (which execute on Facilities nodes 112, FIG. 1). In embodiments that include third party administrators 216, the third party administrators 216 interact with the system 100 via a 3^{rd} Party App 234 (which can execute on Third Party Administrators nodes 106, FIG. 1). Likewise, in embodiments that include Insurance companies 218, the Insurance Companies 218 interact with the system 100 via an Insurance Company App 236 (which can execute on Insurance Company nodes 108, FIG. 1). A scheduling system module 238 executes on the system server 114, FIG. 1 and implements the server side of the methods described below. In some embodiments, the scheduling system module 238 provides a marketplace for the Patients 202 where they can shop for Procedures needed, Treatment Doctors 208, Specialists 210, and Facilities 212,214.

In operation, the Patient 202 sees the Initial Doctor 206 and they determine/select a diagnosis and Procedure. The Initial Doctor 206 enters the Procedure in the Doctor App 224 and sends that to the Patient App 220. The Initial Doctor 206 can then find the Treatment Doctor 208 using the Scheduling System module 238. The Patient 202 can also shop around using the Scheduling System module 238 to find a Treatment Doctor 208 as well. Once the Treatment Doctor 208 has been found, the Procedure information is sent to the Treatment Doctor 208 via the Doctor App 224.

The Treatment Doctor 208 then finds all the needed Specialists 210 and Facilities 212,214 for that Procedure using the Scheduling System module 238. This allows the Treatment Doctor 208 to find and present useful options for the Patient 202 (such as, for example: “this Facility is more expensive but has a slot this week while this other one is only available after 3 weeks”) and give the Patient 202 more flexible choices. Once everything is decided, all the parties agree to a fee schedule for any unanticipated complications resulting from the Procedure.

Once everything is agreed upon and scheduled, the Apps can automatically pay all the Doctors 206,208,210 and Facilities 212,214 that, for example, have automated clearing house (ACH) agreements and generate credit card numbers for ones that do not and let them be paid over the phone, etc.

If there are any additional charges after the Procedure due to complications, then the appropriate provider will be paid directly afterwards through the use of a payment function of the Patient App 220 at a convenient time.

The Employer 204 has access to an aggregation of their Employee's costs via the Employer App 222. Individual costs are not displayed to comport with privacy laws. The Employer App 222 provides reports and graphs that summarize the aggregation of Employee/Patient information.

The Patient App 220 includes a Payment module 240 and a Procedures module 242. The Payment module 240 provides an interface for making an immediate payment. It can be used for manually paying relatively lesser amounts, paying for unexpected complications, or paying for emergencies. The Payment module 240 can be called from the Procedures module 242 in a special manner to make pre-payments for Procedures. The Procedures module 242 provides an interface for keeping track of Procedures for Patients 202, to shop around, to keep track of potential Providers (e.g., doctors 208,210) to do the Procedure, and to communicate with Providers (e.g., doctors 206,208,210) during the process.

Turning now to FIG. 3, the Payment module 240 of the Patient App 220 is described in more detail. Execution of the module 240 begins at 302. Initially, the Patient indicates whether the Procedure is an inpatient procedure or not (304). The term “inpatient procedure” as used herein is intended to indicate a complex, often surgical, procedure that typically requires a Hospital or other medical Facility. Hospitals and Facilities may have a unique way of entering procedure codes. Details are provided below with regard to the Pricing function. If the procedure is an inpatient procedure, the Patient next enters the Inpatient Provider (306).

After identifying the inpatient provider, the Patient will enter the appropriate codes (308). This information will be provided to the Patient by the Provider (e.g., the diagnosing Doctor). If the Procedure is Inpatient, the system will ask for International Statistical Classification of Diseases and Related Health Problems (ICD-10) codes and will find a “diagnosis related group (DRG)” style code using the ICD-10 codes (310) to ultimately determine a Fair Price.

Alternatively, if the procedure is not Inpatient, a non-inpatient provider is identified (312) and then the module 240 asks for (and the price is determined from) Healthcare Common Procedure Coding System (HCPCS, a set of health care procedure codes based on the American Medical Association's (AMA's) Current Procedural Terminology (CPT)) codes (314). All the information is stored to calculate price details for Providers.

The codes will be shown on the bill from the Provider or in documentation from the Doctor. The codes used are based on existing national and industry standards. The Patient also enters the amount charged by the Provider for each item. The module 240 may ask questions or request additional information (316) based on the specific codes, for example, “Was the procedure a special variation?” or “Was the procedure successful?”, that may affect the pricing. The module 240 uses the Provider information and all code information to determine a total Fair Price (318). The module will then display the Fair Price as the amount the Patient App 220 will pay and a Patient Price as the remaining balance for the Patient to pay, if any. The Patient can go back and edit inputs at this point to make any corrections. If the Provider has an ACH agreement in the system, then the Fair Price amount will be paid via ACH to the Provider at this point (320). Otherwise, the module 240 will generate a one-time use credit card number for the Fair Price amount and the Provider charges to the number like any other credit card. Execution of the module 240 completes at 322.

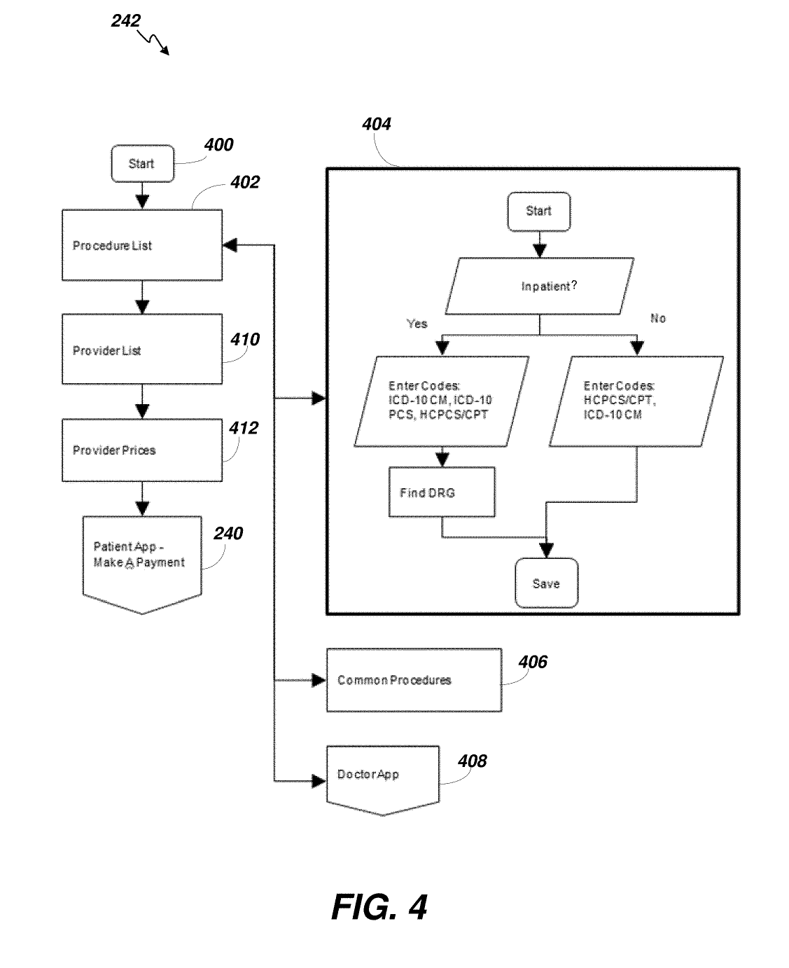

Turning now to FIG. 4, details of the Procedures module 242 are illustrated. Execution of the module 242 starts at 400. The Procedures module 242 provides an interface 402 in the form of a list of all pending Procedures and a menu to access several functions. The interface 402 of the module 242 can also include an option to view historical Procedures including canceled, performed, and paid for Procedures.

A Procedure Edit function 404 allows the Patient to enter the exact medical details of the Procedure with help from the Patient's Doctor. This function 404 is similar to the initial portion of the payment module 240 described above with respect to FIG. 3. The function 404 initially asks whether or not the Procedure is Inpatient. As mentioned above, an Inpatient procedure is typically a complex, often surgical Procedure that will require a Hospital or Facility. As also mentioned above, Hospitals and Facilities may have a unique way of entering procedure codes. Details are provided below with regard to the Pricing function.

After indicating the procedure is an inpatient procedure, the Patient will enter the appropriate codes. This information will be provided to the Patient by the diagnosing Doctor. As above, if the Procedure is Inpatient, the system will ask for International Statistical Classification of Diseases and Related Health Problems (ICD-10) codes and find a “diagnosis related group (DRG)” style code using the ICD-10 codes and determine a Fair Price. If the procedure is not Inpatient, the price is determined from Healthcare Common Procedure Coding System (HCPCS, a set of health care procedure codes based on the American Medical Association's (AMA's) Current Procedural Terminology (CPT)) codes. All the information is stored to calculate price details for Providers.

The module 242 includes a Common Procedures function 406 that provides an interface that presents a list of common Procedures. This function helps avoid requiring the Patient to have go through the full Procedure Edit function for simple Procedures.

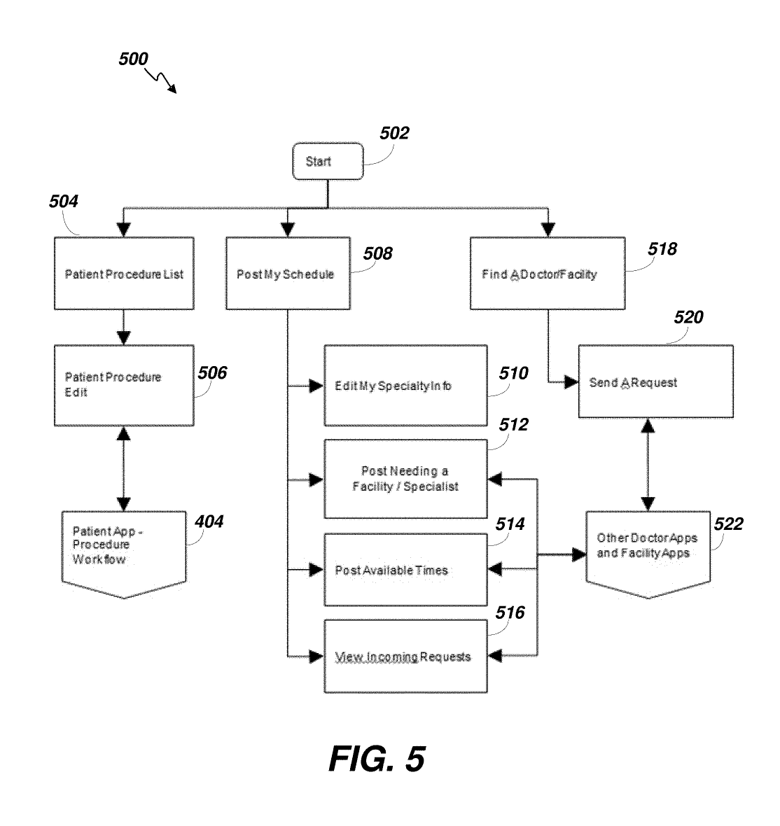

The module 242 also includes a Doctor App function 408 as shown in the Procedures module 242 of the Patient App 220 that allows the Doctor to edit the Procedure using the Doctor App (described below with respect to FIG. 5) executing on the Doctor's Node 102 (FIG. 1) to help the Patient, speed up the process, and reduce errors.

The Procedures module 242 of the Patient App 220 also includes a Provider List function 410 that includes an interface with a ranked listing of all Providers known to the system that are capable of performing the Procedure. The factors that can be used to determine ranking are distance from Patient to the Provider, price, if the price is guaranteed or very consistent, Provider performance rankings and quality measures, if the Provider has relevant offers to the Patient, and if the Provider has sooner available dates. In some embodiments, there will also be nonlinearities or exceptions to the order of the above-listed factors. For example, for expensive procedures, price can be weighted more than distance, while for less expensive procedures, distance can be weighted more than price. In some embodiments, the Patient and/or Doctor can select the priority weights of the ranking factors. All the information will also be appropriately displayed here with useful and exceptional aspects highlighted.

The Procedure module 242 of the Patient App 220 also includes a Provider Prices function 412 which provides an interface that shows the prices for the selected provider. If the Provider has an explicit agreement in place, then the Provider Prices function 412 shows the exact price for this Procedure. If the system has sufficient historical data for this Provider, then estimated prices will be displayed. Otherwise the system will show the Provider's phone number and guide the Patient to call the Provider.

Once the Patient decides on a given provider, the module 242 will create an agreement specifying that any additional charges resulting from complications will be paid according to the same schedule and that the Provider accepting this pre-payment accepts and is bound to this agreement. The system will then copy this information into the Payment module 240 of the Patient App 220 and generate an ACH or credit card payment.

Turning now to FIG. 5, details of the Doctor App 224, 226, 228 are depicted in a flowchart 500. Execution starts at 502. The flowchart 500 of the Doctors App includes a Patient Procedure List function 504 that provides an interface listing all the Procedures that the Doctor's Patients allow the Doctor to view. The Doctor App flowchart 500 includes a Patient Procedure Edit function 506 that allows the Doctor to edit the Patient's Procedure in a very similar manner as the Procedure Edit function 404 in the Procedures module 242 of the Patient App 220. The Doctor can also give additional notes to the Patient which will be displayed in the Patient App 220.

The Doctor App flowchart 500 includes a Post My Schedule function 508 that provides an interface that allows the Doctor to post available services and schedules for both Patients and other Doctors to find.

The Doctor App flowchart 500 includes an Edit My Specialty Information function 510 that provides an interface that allows the Doctor to post their specialty and what the Doctor does in detail. In some embodiments, the Doctor can post in a freeform manner. For example, a Doctor can indicate that the Doctor specializes in a high tech angioplasty and this specialization will be conveyed by the system to people searching for heart specialties.

The Doctor App flowchart 500 includes a Post Needing a Facility/Specialist function 512 that provides an interface that allows the Doctor to post a message indicating a need for a Facility or a Specialist for some given time interval and, in some embodiments, include a price range or offer as well. This function allows Doctors to shop around for their subcontracts and have more independence from the local Hospitals and Facilities.

The Doctor App flowchart 500 includes a Post Available Times function 514 that provides an interface that allows the Doctor, mostly specialists, to post the times the Doctor has available to offer their services and if they want, different fee rates at different times. This helps the Doctors fill their capacity especially if they have equipment or facilities with large maintenance costs.

The Doctor App flowchart 500 includes a View Incoming Requests function 516 that provides an interface that allows the Doctor to view and further communicate with other Doctors who want the Doctor's services. This function allows offers to be easily compared for both price and schedule.

The Doctor App flowchart 500 includes a Find A Doctor/Facility function 518 that provides an interface that allows the Doctor to actively search for services or resources another Doctor or Facility has posted. The Doctor can search for specific specialties, times and prices and view different search results with their schedules compared to the Doctor's schedule. A Send Request function 520 provides an interface that allows the Doctor, once he has selected a facility/doctor, to notify and communicate with the Facility or Specialist to work out details. These notices are displayed on the Doctor's View Incoming Requests function 516 interface. The various functions can communicate with the Doctors Apps of other doctors 522 to facilitate the information exchanges described above.

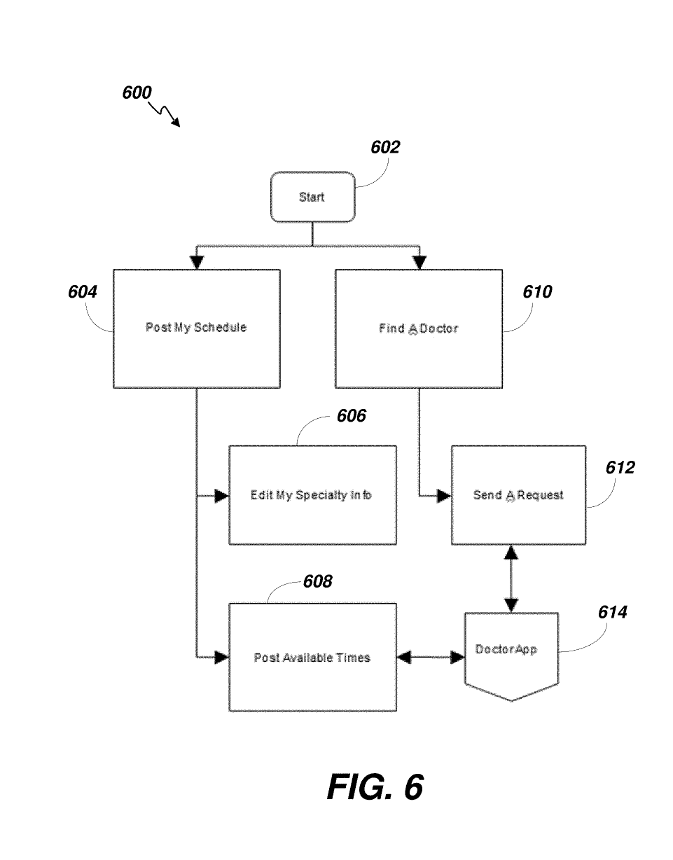

Turning now to FIG. 6, an example embodiment of a Facility App 230,232 is depicted as a flowchart. Execution starts at 602. The Facility App 230,232 can be implemented in a manner similar to the Doctor App flowchart 500 with functions not relevant to Facilities simply removed. Thus, the remaining functions operate as described above with respect to the Doctor's App flowchart 500.

The Facility App 230,232 includes a Post My Schedule function 604 that provides an interface that allows the Facility Doctor/staff to post available services and schedules for both Patients and other Doctors to find.

The Facility App 230,232 includes an Edit My Specialty Information function 606 that provides an interface that allows the Facility Doctor/staff to post their specialty and what the Facility Doctor/staff does in detail. In some embodiments, the Facility Doctor/staff can post in a freeform manner.

The Facility App 230,232 includes a Post Available Times function 608 that provides an interface that allows the Facility Doctor/staff, to post the times the Facility Doctor/staff has available to offer their services and if they want, different fee rates at different times. This helps the Doctors/staff fill their capacity especially if they have equipment or facilities with large maintenance costs.

The Facility App 230,232 includes a Find A Doctor/Facility function 610 that provides an interface that allows the Doctor/staff to actively search for services or resources another Doctor or Facility has posted. The Doctor/staff can search for specific specialties, times and prices and view different search results with their schedules compared to the Doctor's schedule. A Send Request function 612 provides an interface that allows the Doctor/staff, once he has selected a facility/doctor, to notify and communicate with the Facility or Specialist to work out details. These notices can be displayed on a Doctor's View Incoming Requests function interface. The various functions can communicate with the Doctors Apps 224,226,228 and Facility Apps 230,232 of other doctors/facilities 614 to facilitate the information exchanges described above.

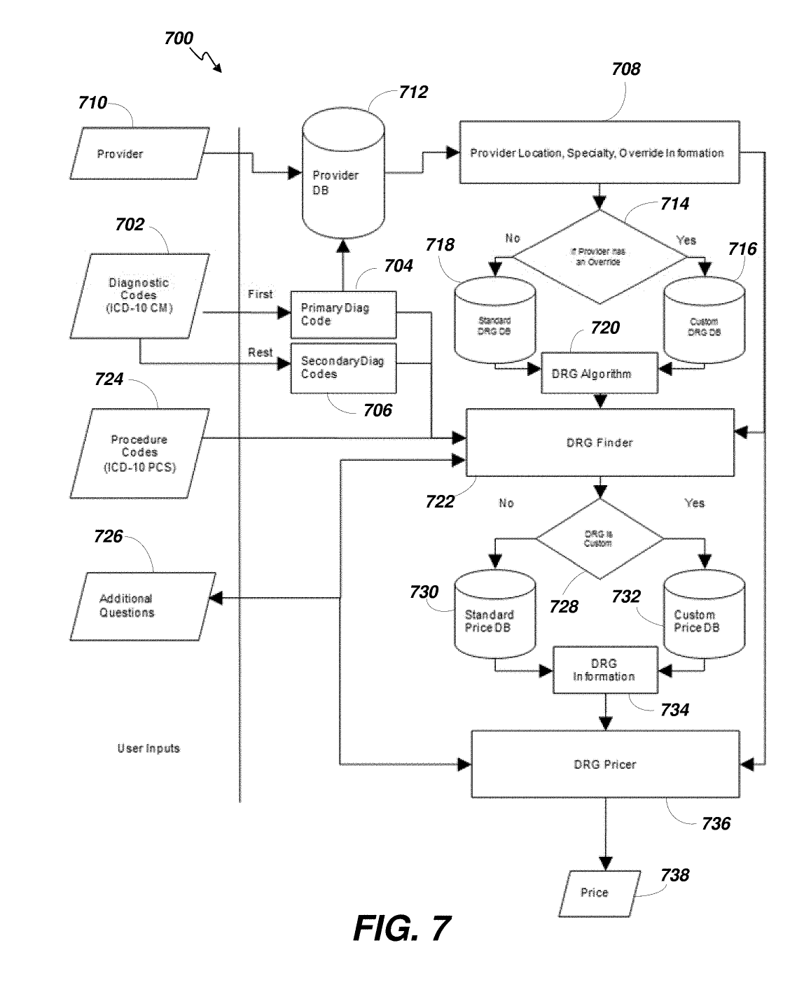

FIG. 7 depicts a flowchart illustrating an example of a Pricing module 700. Embodiments of the system can accommodate two pricing systems: Diagnosis Related Groups (DRG) based pricing and Line Item based pricing. The DRG based pricing encompasses the concept that inpatient procedures are paid as one composite price instead of dozens of line items. This both simplifies the Patient's task of understanding the price of a procedure and allows for easy representation of innovation by Providers. Line Item based pricing supplements DRG pricing and uses prices based on a national standard.

Thus, using DRGs, instead of paying for dozens of line items for a complicated procedure, the payment is just for fixing the base problem. Thus, for example, the Patient will pay a fixed price of $4000 for a concussion or $6000 for a knee surgery instead of $200 for a drug, $150 for nurse time, $200 for the recovery room, and a long list of other costs. In this manner, the Patient has information at a more useful and appropriate level of detail, as it would be extraordinarily difficult to compare dozens of line items that do not quite match for different providers. And since the system can control the fixed price and have it based on national standards, price gouging and adding extraneous line items by Providers is made much more difficult.

However, if a level of modification is needed, the system does allow additional line items to be added to the base DRG price. But since these are explicit additions to the base price and there are fewer of them, the Patient is far more likely to understand the additions and ask the Doctor to justify them, than if the additions buried among dozens of other items.

In some embodiments, a specific system of DRGs can be used that allow for a relatively high degree of customization at the Specialty and Provider level. For example, if a Provider streamlines a surgery process for improved recovery time or builds a small facility that can do the same job as a hospital but for considerably reduced overhead costs, then custom DRGs can be created that are tailored to these improvements, reflect the better prices, and still give the physician the opportunity to determine to use the improvements or the traditional DRG.

In operation the pricing module 700 first splits the Diagnostic Codes (e.g., ICD-10 CM) into one Primary code and zero to many Secondary codes (702). The Primary code 704 is the first one listed and the Secondary codes 706 are the remainder codes. The pricing module 700 then looks up provider information 708 using the provider name 710 (stored in the Provider DB 712) and the Primary Diagnostic Code 704. The Primary code 704 is used because some Providers can have many Specialties as departments and the Primary code 704 indicates which Specialty is relevant.

The provider information 708 includes an indication whether there is an override DRG Algorithm associated with the Provider. The pricing module 700 determines if the provider has an override (714). If so, custom software code to find DRGs will be used to access a custom DRG DB (716). If not, the system will load a standard algorithm to access a standard DRG DB (718). The appropriate algorithm and data are loaded (720) and then the DRG Finder will run the selected algorithm to find an appropriate DRG (722). This process uses both Diagnosis code sets 704,706 and the Procedure code 724; and the system may ask Additional Questions 726 of the Patient and Initial Doctor to find the exact DRG. These are questions that the user interface (UI) presents to the Patient and/or Initial Doctor that are to be answered at that time.

Once the DRG is found (722), flow splits based on whether the DRG is custom (728). The DRG is priced using Standard databases 730 or Custom databases 732 depending on the DRG along with the Provider's location 708 and sometimes additional questions 726 asked by the DRG Pricing function. The DRG information is complied (734) and passed to a DRG Pricer function 736 which determines and outputs a price 738 based on the compiled DRG information 734.

The following example use case is provided for illustrative purposes. Actual implementation of embodiments of the invention can vary substantially from this illustrative example embodiment. Further, the codes and procedures mentioned are merely fictional examples also just for illustrative purposes. In this example, consider a patient with a mild staph infection who comes into see a doctor. The patient is experiencing sharp chest pains. The Primary Diagnosis is a blocked coronary artery and the Secondary Diagnoses are fever and staph infection. The doctor has a choice of treatment: drugs, angioplasty or invasive surgery. The doctor concludes bypass surgery is the best choice and the doctor and patient look at a few Providers as options.

They look at a traditional Provider, the system looks at the Primary Diagnosis which narrows it down to the various heart DRGs, the procedure narrows it to Invasive Heart Surgery Levels 1-3 Complication and the Secondary Diagnoses narrows it to Level 2 Complication. This DRG is then priced based on the statistics of that Provider.

Next the system searches for and finds an angioplasty specialist Provider that offers a new technique that applies to certain patients. The Provider in this example has previously setup these special techniques and price structures and even schedules within their application. The system runs the Custom Algorithm for this Provider and from the fever and infection information determines that it is their Level 2 Complication for two possible procedures and calculates the prices for each. The system then presents to the doctor the traditional open-heart surgery option and these two angioplasty options, each with guaranteed prices and schedules.

An example advantage of the system is that the system is able to very accurately provide comparisons of the very different traditional invasive surgery option and the new angioplasty procedures from a price perspective. The Doctor may not know about the most recent changes in price for this new technique or may not know about the new variations and offers from this specific specialist. For this example Patient's case, because it potentially involves a new procedure, the procedure may be actually more expensive. With this information, the patient and doctor can make the most informed decision, weighing medical pros and cons, as well as price and schedule pros and cons, very precisely.

The system can be implemented to generate several different revenue streams. In some embodiments, a fee is charged per employee per month to self-funded employers. Optionally, to reduce the fee charged, the system can also retain a portion of the claim cost savings. In some embodiments, a health insurance third party administrator is charged a fee so that they can offer the service to their clients. Optionally, to reduce the fee, the system can also retain a portion of the claim cost savings. In some embodiments, a health insurance company is charged a fee so that they can offer the service as their health insurance products. Optionally, to reduce the fee, the system can also retain a portion of the claim cost savings. In some embodiments, the service is combined with an insurance company's plan to reduce claims. Revenue is generated by reducing costs and increasing profits of the insurance carrier. In some embodiments, the service is combined with a stop loss health insurance policy to reduce claims and increase profits of stop loss carrier. In some embodiments, a fee is charged directly to patients to help them shop for care. This can be done in an advisory role where the patient just uses the system to get comparative prices and always pays directly themselves. Alternatively, the patient can open an account with the system operator and the system operator pays out of that account as in other embodiments. Optionally, to reduce the fee, the system can also retain a portion of the claim cost savings. In some embodiments, doctors and health care providers are charged to direct patients to their facilities. In some embodiments, doctors and health care providers are charged to advertise their services in a substantially customized manner to both patients and other doctors. In addition, the doctors and health care providers can also be charged for access to information regard services patients are looking for or using. In some embodiments, the system can generate discounts from doctors and health care providers in exchange for directing patients to their facilities. A portion of the discounts are retained by the system.

Numerous embodiments are described in this disclosure, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments nor a listing of features of the invention that must be present in all embodiments.

The Title (set forth at the beginning of the first page of this disclosure) is not to be taken as limiting in any way as the scope of the disclosed invention(s).

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

The terms “the invention” and “the present invention” and the like mean “one or more embodiments of the present invention.”

A reference to “another embodiment” in describing an embodiment does not imply that the referenced embodiment is mutually exclusive with another embodiment (e.g., an embodiment described before the referenced embodiment), unless expressly specified otherwise.

The terms “including”, “comprising” and variations thereof mean “including but not limited to”, unless expressly specified otherwise.

The terms “a”, “an” and “the” mean “one or more”, unless expressly specified otherwise.

The term “and/or”, when such term is used to modify a list of things or possibilities (such as an enumerated list of possibilities) means that any combination of one or more of the things or possibilities is intended, such that while in some embodiments any single one of the things or possibilities may be sufficient in other embodiments two or more (or even each of) the things or possibilities in the list may be preferred, unless expressly specified otherwise. Thus for example, a list of “a, b and/or c” means that any of the following interpretations would be appropriate: (i) each of “a”, “b” and “c”; (ii) “a” and “b”; (iii) “a” and “c”; (iv) “b” and “c”; (v) only “a”; (vi) only “b”; and (vii) only “c.”

The term “plurality” means “two or more”, unless expressly specified otherwise.

The term “herein” means “in the present disclosure, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device, component or article is described herein, more than one device, component or article (whether or not they cooperate) may alternatively be used in place of the single device, component or article that is described. Accordingly, the functionality that is described as being possessed by a device may alternatively be possessed by more than one device, component or article (whether or not they cooperate).

Similarly, where more than one device, component or article is described herein (whether or not they cooperate), a single device, component or article may alternatively be used in place of the more than one device, component or article that is described. For example, a plurality of computer-based devices may be substituted with a single computer-based device. Accordingly, the various functionality that is described as being possessed by more than one device, component or article may alternatively be possessed by a single device, component or article.

The functionality and/or the features of a single device that is described may be alternatively embodied by one or more other devices that are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Headings of sections provided in this disclosure are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining, recognizing, and the like.

A “display” as that term is used herein is an area that conveys information to a viewer. The information may be dynamic, in which case, an LCD, LED, CRT, Digital Light Processing (DLP), rear projection, front projection, or the like may be used to form the display. The aspect ratio of the display may be 4:3, 16:9, or the like. Furthermore, the resolution of the display may be any appropriate resolution such as 480i, 480p, 720p, 1080i, 1080p or the like. The format of information sent to the display may be any appropriate format such as Standard Definition Television (SDTV), Enhanced Definition TV (EDTV), High Definition TV (HDTV), or the like. The information may likewise be static, in which case, painted glass may be used to form the display. Note that static information may be presented on a display capable of displaying dynamic information if desired. Some displays may be interactive and may include touch screen features or associated keypads as is well understood.

The present disclosure may refer to a “control system” or program. A control system or program, as that term is used herein, may be a computer processor coupled with an operating system, device drivers, and appropriate programs (collectively “software”) with instructions to provide the functionality described for the control system. The software is stored in an associated memory device (sometimes referred to as a computer readable medium). While it is contemplated that an appropriately programmed general purpose computer or computing device may be used, it is also contemplated that hard-wired circuitry or custom hardware (e.g., an application specific integrated circuit (ASIC)) may be used in place of, or in combination with, software instructions for implementation of the processes of various embodiments. Thus, embodiments are not limited to any specific combination of hardware and software.

A “processor” means any one or more microprocessors, Central Processing Unit (CPU) devices, computing devices, microcontrollers, digital signal processors, or like devices. Exemplary processors are the INTEL PENTIUM or AMD ATHLON processors.

The term “computer-readable medium” refers to any statutory medium that participates in providing data (e.g., instructions) that may be read by a computer, a processor or a like device. Such a medium may take many forms, including but not limited to non-volatile media, volatile media, and specific statutory types of transmission media. Non-volatile media include, for example, optical or magnetic disks and other persistent memory. Volatile media include DRAM, which typically constitutes the main memory. Statutory types of transmission media include coaxial cables, copper wire and fiber optics, including the wires that comprise a system bus coupled to the processor. Common forms of computer-readable media include, for example, a floppy disk, a flexible disk, hard disk, magnetic tape, any other magnetic medium, a CD-ROM, Digital Video Disc (DVD), any other optical medium, punch cards, paper tape, any other physical medium with patterns of holes, a RAM, a PROM, an EPROM, a FLASH-EEPROM, a USB memory stick, a dongle, any other memory chip or cartridge, a carrier wave, or any other medium from which a computer can read. The terms “computer-readable memory” and/or “tangible media” specifically exclude signals, waves, and wave forms or other intangible or non-transitory media that may nevertheless be readable by a computer.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols. For a more exhaustive list of protocols, the term “network” is defined below and includes many exemplary protocols that are also applicable here.

It will be readily apparent that the various methods and algorithms described herein may be implemented by a control system and/or the instructions of the software may be designed to carry out the processes of the present invention.

Where databases are described, it will be understood by one of ordinary skill in the art that (i) alternative database structures to those described may be readily employed, and (ii) other memory structures besides databases may be readily employed. Any illustrations or descriptions of any sample databases presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models, hierarchical electronic file structures, and/or distributed databases) could be used to store and manipulate the data types described herein. Likewise, object methods or behaviors of a database can be used to implement various processes, such as those described herein. In addition, the databases may, in a known manner, be stored locally or remotely from a device that accesses data in such a database. Furthermore, while unified databases may be contemplated, it is also possible that the databases may be distributed and/or duplicated amongst a variety of devices.

As used herein a “network” is an environment wherein one or more computing devices may communicate with one another. Such devices may communicate directly or indirectly, via a wired or wireless medium such as the Internet, LAN, WAN or Ethernet (or IEEE 802.3), Token Ring, or via any appropriate communications means or combination of communications means. Exemplary protocols include but are not limited to: Bluetooth™, Time Division Multiple Access (TDMA), Code Division Multiple Access (CDMA), Global System for Mobile communications (GSM), Enhanced Data rates for GSM Evolution (EDGE), General Packet Radio Service (GPRS), Wideband CDMA (WCDMA), Advanced Mobile Phone System (AMPS), Digital AMPS (D-AMPS), IEEE 802.11 (WI-FI), IEEE 802.3, SAP, the best of breed (BOB), system to system (S2S), or the like. Note that if video signals or large files are being sent over the network, a broadband network may be used to alleviate delays associated with the transfer of such large files, however, such is not strictly required. Each of the devices is adapted to communicate on such a communication means. Any number and type of machines may be in communication via the network. Where the network is the Internet, communications over the Internet may be through a website maintained by a computer on a remote server or over an online data network including commercial online service providers, bulletin board systems, and the like. In yet other embodiments, the devices may communicate with one another over RF, cable TV, satellite links, and the like. Where appropriate encryption or other security measures such as logins and passwords may be provided to protect proprietary or confidential information.

Communication among computers and devices may be encrypted to insure privacy and prevent fraud in any of a variety of ways well known in the art. Appropriate cryptographic protocols for bolstering system security are described in Schneier, APPLIED CRYPTOGRAPHY, PROTOCOLS, ALGORITHMS, AND SOURCE CODE IN C, John Wiley & Sons, Inc. 2d ed., 1996, which is incorporated by reference in its entirety.

It will be readily apparent that the various methods and algorithms described herein may be implemented by, e.g., appropriately programmed general purpose computers and computing devices. Typically a processor (e.g., one or more microprocessors) will receive instructions from a memory or like device, and execute those instructions, thereby performing one or more processes defined by those instructions. Further, programs that implement such methods and algorithms may be stored and transmitted using a variety of media (e.g., computer readable media) in a number of manners. In some embodiments, hard-wired circuitry or custom hardware may be used in place of, or in combination with, software instructions for implementation of the processes of various embodiments. Thus, embodiments are not limited to any specific combination of hardware and software. Accordingly, a description of a process likewise describes at least one apparatus for performing the process, and likewise describes at least one computer-readable medium and/or memory for performing the process. The apparatus that performs the process can include components and devices (e.g., a processor, input and output devices) appropriate to perform the process. A computer-readable medium can store program elements appropriate to perform the method.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

The foregoing description discloses only example embodiments of the invention. Modifications of the above-disclosed apparatus, systems and methods which fall within the scope of the invention will be readily apparent to those of ordinary skill in the art.

Accordingly, while the present invention has been disclosed in connection with exemplary embodiments thereof, it should be understood that other embodiments may fall within the spirit and scope of the invention, as defined by the following claims.

The invention claimed is:

1. A system for providing healthcare service comprising:
a patient device including a processor coupled to a memory and a communications capability, the memory storing instructions executable by the processor, the instructions adapted to:
provide a payment module including instructions to:
provide an interface for indicating whether a procedure is an inpatient procedure;
provide an interface for entering codes and prices for the procedure;
provide an interface for asking questions of the patient;
create a payment summary; and
generate a payment; and
provide a procedures module including instructions to:
provide an interface listing pending procedures,
provide an interface for editing procedure descriptions;
provide an interface listing providers that can perform a particular procedure; and
provide an interface for displaying prices of services of a selected provider;
a doctor device including a processor coupled to a memory and a communications capability, the memory storing instructions executable by the processor, the instructions adapted to:
provide an interface displaying a listing of procedures of patients of a doctor;
provide an interface for posting the doctor's available services and schedules;
provide an interface for editing the doctor's specialty;
provide an interface for posting needs for facilities and specialists;
provide an interface for viewing incoming requests for services;
provide an interface for searching for facilities and specialists; and
provide an interface for communicating with facilities and specialists; and
a server including a processor coupled to a memory and a communications capability, the memory storing instructions executable by the processor, the instructions adapted to:
facilitate communications between the patent device and the doctor device;
provide a first database for storing information communicated between the patent device and the doctor device;
provide a second database for storing provider information;
provide a third database for storing information regarding diagnosis related groups (DRGs) of procedures;
provide a forth database for storing information regarding procedure pricing; and
determine pricing based on information stored in the first through fourth databases.
